(19) United States
(12) Patent Application Publication
(10) Pub. No.: US 2017/0319539 A1
(43) Pub. Date: Nov. 9, 2017

(54) Amorphous Empagliflozin

(71) Applicant: Mylan Laboratories LTD., Hyderabad (IN)

(72) Inventors: Ramakoteswara Rao Jetti, Hyderabad (IN); Aggi Ramireddy Bommareddy, Hyderabad (IN); Amit Singh, Hyderabad (IN); Vijaya Krishna Ravi, Hyderabad (IN); Subramanyam Dandala, Hyderabad (IN)

(73) Assignee: Mylan Laboratories LTD., Hyderabad (IN)

(21) Appl. No.: 15/515,787

(22) PCT Filed: Oct. 1, 2015
(86) PCT No.: PCT/IB2015/057513

§ 371 Date: Mar. 30, 2017

(30) Foreign Application Priority Data
Oct. 1, 2014 (IN) 4964/CHE/2014

(51) Int. Cl. A61K 31/351; A61K 45/06; A61K 31/155; A61K 31/522; C07D 407/12; A61K 47/40

(52) U.S. Cl. CPC A61K 31/351; C07D 407/12; A61K 47/40; A61K 31/155; A61K 31/522; A61K 45/06

(57) ABSTRACT

The present disclosure relates to solid dispersion of amorphous empagliflozin and its process thereof.

### CROSS-REFERENCE TO RELATED APPLICATIONS

This application claims the benefit of Indian provisional patent application No. 4964/CHE/2014, filed on Oct. 1, 2014, which is hereby incorporated by reference in its entirety.

### BACKGROUND OF THE INVENTION

### Field of the Disclosure

The present disclosure relates to amorphous forms of empagliflozin and processes for preparing the same.

### Background of the Disclosure

Empagliflozin is a novel. SGLT2 (sodium/glucose cotransporter-2) inhibitor that is described for the treatment or improvement in glycemic control in patients with type 2 diabetes mellitus.

JARDIANCE® tablets contains empagliflozin and chemically known as (1S)-1,5-anhydro-1-C-[4-chloro-3-[[4-[[(3 S)-tetrahydro-3-furanyl]oxy]phenyl]methyl] phenyl]-D-glucitol (Formula I).

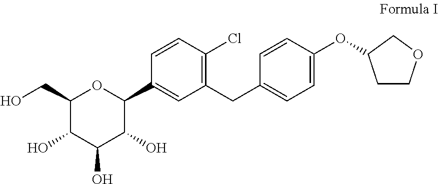

U.S. Pat. No. 7,713,938 discloses stable a crystalline form of empagliflozin and a pharmaceutical composition or medicament comprising the crystalline form. Additional crystalline forms of empagliflozin are disclosed in U.S. Pat. No. 7,723,309 and U.S. Pat. No. 8,802,842.

### SUMMARY OF THE DISCLOSURE

One aspect of the present disclosure is to provide amorphous forms of empagliflozin. In one embodiment, the amorphous form of empagliflozin is provided as an amorphous solid dispersions of empagliflozin, for example, with a pharmaceutically acceptable polymer (e.g. polyvinylpyrrolidinone). In other embodiments, the empagliflozin is complexed with a pharmaceutically acceptable carrier. The pharmaceutically acceptable carrier may be a α-cyclodextrin, a β-cyclodextrin, or a γ-cyclodextrin. In some embodiments, the pharmaceutically acceptable carrier is a hydroxypropyl-β-cyclodextrin. Such combinations with cyclodextrin carriers can provide amorphous empagliflozin complexes. For example, powder X-ray diffraction (PXRD) patterns for certain amorphous empagliflozin complex are shown in FIG. 1 (complex with β-cyclodextrin) and FIG. 2 (complex with hydroxypropyl β-cyclodextrin).

Another aspect of the present disclosure is to provide a process for preparing an amorphous empagliflozin complex comprising the steps of:
a) dissolving empagliflozin in a solvent;
b) adding a cyclodextrin; and
c) isolating an amorphous empagliflozin complex.

Yet another aspect of the present disclosure is to provide a process for preparing an amorphous empagliflozin complex comprising the steps of:
a) dissolving empagliflozin in a solvent to provide a first solution;
b) dissolving a cyclodextrin in water to provide a second solution;
c) combining the first solution and the second solution (e.g., adding the second solution to the first solution) to provide a combined solution; and
d) isolating an amorphous empagliflozin complex from the combined solution.

Yet another aspect of the present disclosure is to provide a process for preparing an amorphous empagliflozin comprising the steps of;
a) dissolving empagliflozin in an organic solvent, and
b) removing the solvent to obtain an amorphous empagliflozin.

Another aspect of the present disclosure is to an amorphous empagliflozin.

### BRIEF DESCRIPTION OF THE FIGURES

FIG. 1 is a representative powder x-ray diffraction (PXRD) pattern of an amorphous empagliflozin complex with β-cyclodextrin, prepared according to Example 5 & 6.

FIG. 2 is a representative powder x-ray diffraction (PXRD) pattern of an amorphous empagliflozin complex with hydroxypropyl β-cyclodextrin, prepared according to Example 7 & 8.

FIG. 3 is a representative powder x-ray diffraction (PXRD) pattern of an amorphous empagliflozin solid dispersion with plasdone-S-630.

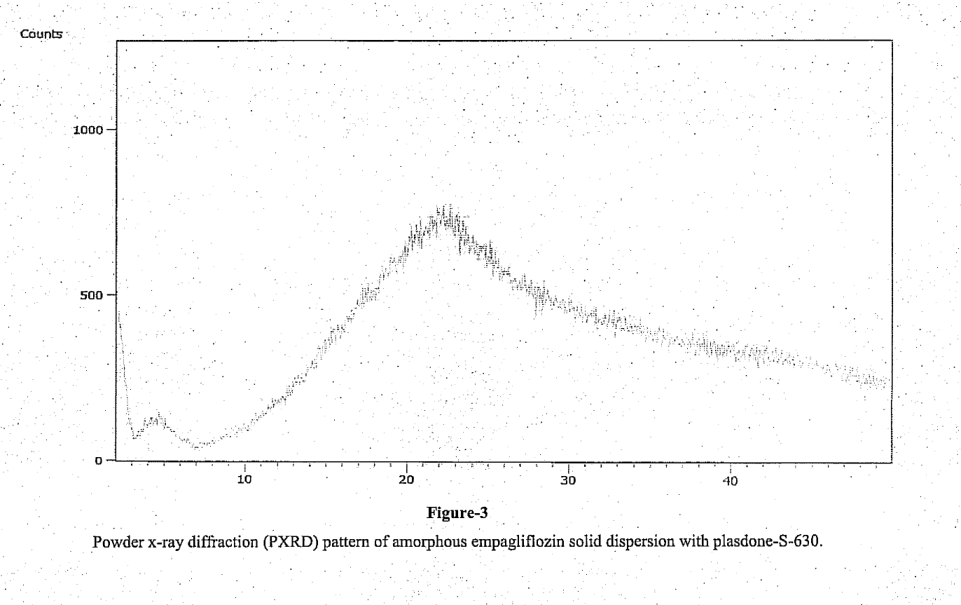

FIG. 4 is a representative powder x-ray diffraction (PXRD) pattern of an amorphous empagliflozin.

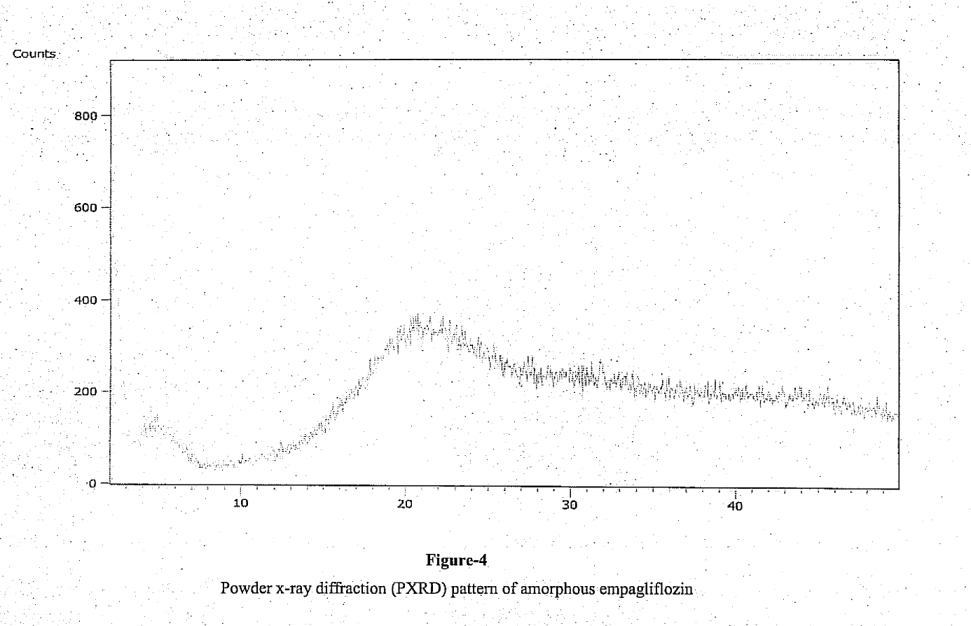

### INSTRUMENTATION

Powder X-Ray Diffraction (PXRD)

The amorphous forms (e.g., solid dispersions and complexes) of empagliflozin of the present disclosure are characterized by X-ray powder diffraction patterns. Thus, the X-ray diffraction patterns of the present disclosure were measured on BRUKER D-8 Discover powder diffractometer equipped with goniometer of θ/2θ configuration and Lynx Eye detector. The Cu-anode X-ray tube was operated at 40 kV and 30 mA. The experiments were conducted over the 2θ range of 2.0°-50.0°, 0.030° step size and 0.4 seconds step time.

### DETAILED DESCRIPTION OF THE DISCLOSURE

It is to be understood that the description of the present invention has been simplified to illustrate elements that are relevant for a clear understanding of the invention, while eliminating, for purposes of clarity, other elements that may be well known.

The present disclosure relates to process for the preparation of amorphous forms of empagliflozin and process for preparing the same. In certain embodiments, empagliflozin is dissolved in an organic solvent and the amorphous form of empaglifiozin is isolated from the solution.

One embodiment of the present disclosure provides an amorphous forms of empagliflozin as an amorphous solid dispersions.

In another embodiment, the present disclosure provides an amorphous form of empagliflozin as an amorphous empagliflozin complex.

Within the context of the present disclosure, and without being limited by any one theory of operation, a solid dispersion may be a molecular dispersion of a compound, particularly a drug substance within a carrier matrix. Formation of a molecular dispersion provides a means of reducing the particle size, in some embodiments, to molecular levels. As the carrier dissolves, the drug can be exposed to the dissolution media as fine particles that can be amorphous. The fine particles dissolve and may absorb more rapidly than larger particles.

The term “solid dispersion” as used herein, refers to a system in a solid state including at least two components, wherein one component is dispersed (e.g., homogeneously), throughout the other component or components.

The term “amorphous solid dispersion” as used herein, refers to stable solid dispersions comprising amorphous drug substance and a carrier matrix.

An “amorphous drug substance” as used herein, is an amorphous solid dispersion containing drug substance in a substantially amorphous solid state form. A substantially amorphous state may include at least about 80%, at least about 90%, or at least 95% of the drug substance in the dispersion is in an amorphous form. In one particular embodiment, a substantially amorphous state includes at least about 95% of the drug substance in the dispersion in an amorphous form. Whether a drug substance in the dispersion is in an amorphous form can be characterized, for example, by powder x-ray diffraction techniques as described herein or otherwise known to those skilled in the art.

The term “amorphous complex” as used herein refers to a composition comprising a drug substance and a complexing agent, where the drug substance in the composition is in the amorphous state as determined by PXRD. “Complexing agent” as used herein refers to a compound capable of forming a non-covalent complex with the drug substance (e.g., host-guest and/or a guest-host interactions to form an inclusion complex with the drug substance). Examples of complexing agents include cyclodextrins, such as those noted herein. In such complexes, at least a portion of the drug substance may be or is complexed with the complexing agent. For example, where the complexing agent is a cyclodextrin e.g., hydroxypropyl β-cyclodextrin), at least a portion of the drug substance within the composition may be or is complexed with the cyclodextrin (e.g., at least a portion of the drug substance within the composition may or does form an inclusion complex with the cyclodextrin).

The term “about” as used herein means +/−10% or less of the noted value. In certain embodiments, “about” means +/−10%; or +/−5% of the noted value.

Another embodiment of the present disclosure is to provide a process for preparing an amorphous empagliflozin complex comprising the steps of:
a) dissolving empagliflozin in a solvent;
b) adding a cyclodextrin; and
c) isolating an amorphous empagliflozin complex.

According to the present embodiment, empagliflozin is dissolved in solvent at an elevated temperature. The cyclodextrin is added to the solution at same temperature, stirred to get clear solution at same temperature and the resulting solution is cooled to room temperature. Solvent is removed to obtain an amorphous empagliflozin complex comprising, or consisting essentially of, or consisting of, empagliflozin and the cyclodextrin.

Yet another embodiment of the present disclosure is to provide a process for preparing an of amorphous empagliflozin complex comprising the steps of:
a) dissolving empagliflozin in a solvent to provide a first solution;
b) dissolving a cyclodextrin in water to provide a second solution;
c) combining the first solution and second solution (e.g., adding the second solution to the first solution) to provide a combined solution; and
d) isolating amorphous empagliflozin complex from the combined solution.

According to the present embodiment, empagliflozin is dissolved in a solvent at an elevated temperature. In certain embodiments, the cyclodextrin is dissolved in water and the aqueous solution is added to the empagliflozin solution at same elevated temperature to get a clear solution. The resulting solution is cooled to room temperature. The solvent is removed from the combined solution to obtain an amorphous empagliflozin complex comprising, or consisting essentially of, or consisting of, empagliflozin and the cyclodextrin.

Yet another embodiment of the present disclosure is to provide a process for the preparation of an amorphous empagliflozin comprising the steps of;
a) dissolving empagliflozin in an organic solvent; and
b) removing the solvent to obtain an amorphous empaglifiozin.

According to the present embodiment, empagliflozin is dissolved in organic solvent at elevated temperature, the resulting solution is cooled and subjected to particle-free filtration. Solvent is removed from the clear filtrate to obtain an amorphous empagliflozin.

As used herein, the term “solvent” unless otherwise indicated, refers to an alcohol solvent, ketone solvent, chlorinated solvent, water or a mixture thereof.

As used herein, alcohol solvent include, but are not limited to methanol, ethanol, isopropanol or mixtures thereof; ketone solvent include, but are not limited to acetone or methyl ethyl, ketone; and chlorinated solvents include, but are not limited to dichloromethane or chloroform.

According to the present embodiment, the solvent is removed by known techniques include, but are not limited to, evaporation, distillation, spray drying, filtration, lyophilization, or by using an agitated thin film drier (ATFD).

As used herein, the term “elevated temperature” unless otherwise indicated, refers to reflux temperature (i.e., boiling point) of the solvent employed.

As used herein, the term “room temperature” unless otherwise indicated, refers to about 20-35° C. In certain embodiments, “room temperature” indicates about 25-30° C.

According to the present disclosure the starting material empagliflozin may be crystalline, amorphous, or semi-solid in nature.

The term “hydroxypropyl-β-cyclodextrin” as used herein refers to a β-cyclodextrin substituted with 2-hydroxypropyl groups at available hydroxyl groups of the glucose units within the β-cyclodextrin [e.g., CAS no 128446-35-5]. For example, hydroxypropyl-β-cyclodextrin may refer to a β-cyclodextrin having an average degree of substitution of about 0.1-2.0, or about 0.5-2.0, or about 0.5-1.3 units of 2-hydroxypropyl groups per glucose unit. Suitable examples include, but are not limited to hydroxypropyl-β-cyclodextrins available from Sigma-Aldrich Co. (St. Louis, Mo.; e.g., Cat. No. H107, average degree of substitution is 0.5-1.3 unit of 2-hydroxypropyl groups per glucose unit; Cat. No. Y0000186, “hydroxypropylbetadex”; Cat. No. 332593 (average molar substitution=0.6); Cat. No. 332607 (average molar substitution=0.8); Cat No. 389145 (average molar substitution=1.0)).

Yet another embodiment is to provide a pharmaceutical composition comprising amorphous empagliflozin.

Yet another embodiment is to provide a pharmaceutical composition comprising an amorphous empagliflozin solid dispersion or an amorphous empagliflozin complex.

In accordance with one embodiment of the present disclosure, the dissolution properties of drugs may be improved by their conversion to an amorphous state or by complexation with cyclodextrins. The present disclosure provides a pharmaceutical composition including a solid dispersion which may be prepared by dissolving a water-insoluble drug and a substituted cyclodextrin in an organic solvent with or without water to make a mixture. The mixture may then be dried under a reduced pressure or spray dried.

According to the present disclosure, the preparation of empagliflozin complexed with a α-cyclodextrin, a β-cyclodextrin, a γ-cyclodextrin, with some embodiments employing a hydroxypropyl-β-cyclodextrin, may be achieved in the following manner. Initially empagliflozin and the cyclodextrin may be dissolved in a solvent. The empagliflozin and the cyclodextrin (e.g., hydroxypropyl-β-cyclodextrin) can be dissolved at a weight ratio of about 20:1 to about 1:1 w/w empagliflozin:cyclodextrin; or about 15:1 to about 1:1, or about 15:1 to about 2:1, or about 10:1 to about 2:1. In certain embodiments, the empagliflozin and the cyclodextrin (e.g., hydroxypropyl-β-cyclodextin) can be dissolved at a weight ratio of about 10:1 to about 8:1. In certain embodiments, the empagliflozin and the cyclodextrin (e.g., hydroxypropyl-β-cyclodextrin) can be dissolved at a weight ratio of about 4:1 to about 2:1. In certain other embodiments, the empagliflozin and the cyclodextin (e.g., hydroxypropyl-β-cyclodextrin) can be dissolved at a weight ratio of about 9:1. In certain embodiments, the empagliflozin and the cyclodextrin (e.g., hydroxypropyl-β-cyclodextrin) can be dissolved at a weight ratio of about 11.

The solvent may then be removed, yielding solids which could be directly compressed to tablets that dissolve completely within minutes when ingested by patients. Amorphous water soluble derivatives of cyclodextrins are potent, nontoxic solubilizers of drugs and

Another aspect of the present disclosure provides a pharmaceutical composition including an amorphous empagliflozin solid dispersion or an amorphous empagliflozin complex (each as described here) and at least one pharmaceutically acceptable carrier, which may be formulated into tablets, capsules, suspensions, dispersions, injectables, or other pharmaceutical forms.

In some embodiments, the amorphous solid dispersions and amorphous complexes of empagliflozin of the present disclosure may be included in tablets for oral administration. One of skill in the art will recognize a wide variety of pharmaceutically acceptable excipients that may be included in such a tablet formulation, including lactose monohydrate, microcrystalline cellulose, croscarmelose sodium, hydroxypropyl cellulose, sodium lauryl sulfate, and magnesium stearate.

Such pharmaceutical composition (e.g., tablets) will generally include an effective amount of empagliflozin for the intended use. In certain embodiments, such pharmaceutical composition (e.g., tablets) include an effective amount of empagliflozin for the treatment of diabetes (e.g., improve glycemic control) in a human patient in need of such treatment (e.g., a patient having type 2 diabetes mellitus; such as an adult having type 2 diabetes mellitus). For example, the pharmaceutical composition (e.g., tablets) may include about 1 mg to about 50 mg; or about 5 mg to about 50 mg; or about 10 mg to about 50 mg; or about 10 mg to about 30 mg; or about 10 mg to about 25 mg of empagliflozin. In certain particular embodiments, the pharmaceutical composition (e.g., tablets) include about 10 mg or about 12.5 mg, or about 15 mg, or about 17.5 mg, or about 20 mg, or about 22.5 mg, or about 25 mg of empagliflozin.

In other embodiments, the pharmaceutical composition (e.g., tablets) of the preceding paragraph may further include an additional therapeutic agent, such as, but not limited to an effective amount of a second antihyperglycemic drug (e.g., useful for the management of type 2 diabetes).

The second antihyperglycemic drug can be, for example, dipeptidyl peptidase-4 (DPP-4) inhibitors. Examples of DPP-4 inhibitors include, but are not limited to, sitagliptin, vildagliptin, saxagliptin, linagliptin, anagliptin, teneligliptin, alogliptin, trelagliptin, gemigliptin, and dutogliptin. In certain embodiments, the second antihyperglycemic drug is linagliptin. An effective amount of linagliptin can be, for example, about 1 mg to about 20 mg; or about 1 mg to about 10 mg; or about 2.5 mg to about 10 mg; or about 2.5 mg to about 5 mg. Examples of combination pharmaceutical composition (e.g., tablets) include, but are not limited to 10 mg of empaglifiozin and 5 mg or linagliptin (“10/5”); or 25 mg of empagliflozin and 5 mg or linagliptin (“25/5”).

In other embodiments, the second antihyperglycemic drug can be a biguanide. Examples of suitable biguanides include metformin, and pharmaceutically acceptable metformin salts, such as metformin hydrochloride. An effective amount of metformin hydrochloride can be, for example, about 100 mg to about 2000 mg, or about 100 mg to about 1500 mg, or about 250 mg to about 1500 mg, or about 250 mg to about 1000 mg, or about 500 mg to about 1000 mg. In particular examples, an effective amount can be 500 mg or 1000 mg. Examples of combination pharmaceutical composition (e.g., tablets) include, but are not limited to, tablets that contain 5 mg empagliflozin and 500 mg metformin hydrochloride; or 5 mg empagliflozin and 1000 mg metformin hydrochloride; or 12.5 mg empagliflozin and 500 mg metformin hydrochloride; or 12.5 mg empagliflozin and 1000 mg metformin hydrochloride.

Certain specific aspects and embodiments of the present application will be explained in greater detail with reference to the following examples, which are provided only for purposes of illustration and should not be construed as limiting the scope of the disclosure in any manner. Reasonable variations of the described procedures are intended to be within the scope of the present application. While particular aspects of the present application have been illustrated and described, it would be apparent to those skilled in the art that various other changes and modifications can be made without departing from the spirit and scope of the disclosure. It is therefore intended to encompass all such changes and modifications that are within the scope of this disclosure.

Solid State Stability

In yet another embodiment, the amorphous empagliflozin, the amorphous empagliflozin solid dispersion, and the amorphous empagliflozin complexes prepared according to the present disclosure have HPLC purity of more than 99.0%.

The physical and chemical stability of amorphous empagliflozin and amorphous empagliflozin forms (e.g., solid dispersions and complexes) were tested by storing the samples at 40° C./75% relative humidity (RH), 25° C./60% RH and at 5±3° C. for 6 months. The samples were analyzed by PXRD and HPLC for final purity. The results are shown in Tables 1-3. The stability data shows the PXRD pattern remains same as initial and there is no degradation observed in HPLC up to six months. This indicates amorphous empagliflozin and amorphous empagliflozin forms are physically and chemically stable.

TABLE 1

| | Polymorph | | | | | |
|---|---|---|---|---|---|---|
| | Amorphous empagliflozin solid dispersion with Plasdone S-630 (ratios) | | | | | |
| | 1:1 | | 1:0.5 | | 1:0.25 | |
| | HPLC | | HPLC | | HPLC | |
| | Purity | | Purity | | Purity | |
| Condition | (%) | PXRD | (%) | PXRD | (%) | PXRD |

at 40° C./75% RH

| | | | | | | |
|---|---|---|---|---|---|---|
| Initial | 99.0 | Amorphous | 99.05 | Amorphous | 99.0 | Amorphous |
| 2 Months | 99.0 | Amorphous | 99.1 | Amorphous | 99.05 | Amorphous |
| 3 Months | 98.9 | Amorphous | 98.9 | Amorphous | 99.05 | Amorphous |
| 6 months | 99.06 | Amorphous | 99.1 | Amorphous | 99.17 | Amorphous |

at 25° C./60% RH

| | | | | | | |
|---|---|---|---|---|---|---|
| Initial | 99.0 | Amorphous | 99.05 | Amorphous | 99.0 | Amorphous |
| 2 Months | 99.04 | Amorphous | 98.9 | Amorphous | 99.11 | Amorphous |
| 3 Months | 99.0 | Amorphous | 99.0 | Amorphous | 99.0 | Amorphous |
| 6 months | 99.06 | Amorphous | 99.11 | Amorphous | 99.18 | Amorphous |

at 5 ± 3° C.

| | | | | | | |
|---|---|---|---|---|---|---|
| Initial | 99.0 | Amorphous | 99.05 | Amorphous | 99.0 | Amorphous |
| 2 Months | 99.04 | Amorphous | 99.13 | Amorphous | 99.11 | Amorphous |
| 3 Months | 98.9 | Amorphous | 99.0 | Amorphous | 99.0 | Amorphous |
| 6 months | 98.9 | Amorphous | 99.11 | Amorphous | 99.12 | Amorphous |

TABLE 2

| | Polymorph | | | |
|---|---|---|---|---|
| | Amorphous | | Amorphous | |
| | empagliflozin | | empagliflozin | |
| | complex with 10% w/w | | complex with 25% w/w | |
| | Hydroxypropyl-β- | | Hydroxypropyl-β- | |
| | cyclodextrin | | cyclodextrin | |
| | HPLC | | HPLC | |
| Condition | Purity (%) | PXRD | Purity (%) | PXRD |

at 40° C./75% RH

| | | | | |
|---|---|---|---|---|
| Initial | 99.26 | Amorphous | 99.29 | Amorphous |
| 2 Months | 99.23 | Amorphous | 99.21 | Amorphous |
| 3 Months | 99.26 | Amorphous | 99.29 | Amorphous |
| 6 months | 99.18 | Amorphous | 99.27 | Amorphous |

TABLE 3

| | Polymorph | | | | | |
|---|---|---|---|---|---|---|
| | | | | | Amorphous solid | |
| | Amorphous | | Amorphous | | dispersion with | |
| | complex with | | complex with | | 10% w/w Povidone K- | |
| | 10% w/w BCD | | 25% w/w BCD | | 30 | |
| | HPLC | | HPLC | | HPLC | |
| | Purity | | Purity | | Purity | |
| Condition | (%) | PXRD | (%) | PXRD | (%) | PXRD |

at 40° C./75% RH (BCD = β-cyclodextrin)

| | | | | | | |
|---|---|---|---|---|---|---|
| Initial | 99.04 | Amorphous | 99.14 | Amorphous | 99.28 | Amorphous |
| 2 Months | 99.21 | Amorphous | 99.18 | Amorphous | 99.11 | Amorphous |
| 3 Months | 99.29 | Amorphous | 99.26 | Amorphous | 99.18 | Amorphous |
| 6 months | 99.24 | Amorphous | 99.3 | Amorphous | 99.38 | Amorphous |

The following examples are provided for illustrative purposes only and are not intended to limit the scope of the invention in anyway.

### EXAMPLES

### Example 1

Empagliflozin (5 g) was dissolved in methanol (100 mL) at 60±5° C. and the clear solution was cooled to 25-30° C. The clear solution was filtered through hyflo to remove any undissolved particulate and subjected to spray drying in a laboratory Spray Dryer (Model Buchi-290) with a solution feed rate of 10 mL/min and an inlet temperature at 70° C. to yield amorphous empagliflozin.

Yield: 66%.

### Example 2

Empagliflozin (10 g) was dissolved in methanol (150 mL) at 60±5° C. and the clear solution was cooled to 25-30° C. In another flask Plasdone S-630 (10 g) was dissolved in methanol (50 mL) at 25-30° C. and the obtained solution was added to the empagliflozin solution at 25-30° C. The resulting solution was filtered through hyflo to remove any undissolved particulate and the clear solution was subjected to spray drying in a laboratory Spray Dryer (Model Buchi-290) with a solution feed rate of 10 mL/min and an inlet temperature at 70° C. to yield pure amorphous empagliflozin solid dispersion.

Yield: 65%.

### Example 3

Empagliflozin (10 g) was dissolved in methanol (150 mL) at 60±5° C. and the clear solution was cooled to 25-30° C. In another flask Plasdone S-630 (5 gm) was dissolved in methanol (50 mL) at 25-30° C. and the obtained solution was added to the empagliflozin solution at 25-30° C. The resulting solution was filtered through hyflo to remove any undissolved particulate and the clear solution was subjected to spray drying in a laboratory Spray Dryer (Model Buchi-290) with a solution feed rate of 10 mL/min and an inlet temperature at 70° C. to yield pure amorphous empagliflozin solid dispersion.

Yield: 68%

### Example 4

Empagliflozin (10 g) was dissolved in methanol (150 mL) at 60±5° C. and the clear solution was cooled to 25-30° C. In another flask, Plasdone S-630 (2.5 g) was dissolved in methanol (50 mL) at 25-30° C. and the obtained solution was added to the empagliflozin solution at 25-30° C. The resulting clear solution was filtered through hyflo to remove any undissolved particulate and subjected to spray drying in a laboratory Spray Dryer (Model Buchi-290) with a solution feed rate of 10 ml/min and an inlet temperature at 70° C. to yield pure amorphous empagliflozin solid dispersion.

Yield: 80%.

### Example 5

Empagliflozin (20 g) was dissolved in methanol (400 mL) at 50±5° C. and hydroxypropyl β-cyclodextrin (2.2 g) was added at same temperature and the mass was maintained for 10 minutes to provide a clear solution. After cooling to 25±5° C., the resulting clear solution was filtered through hyflo to remove any undissolved particulate and subjected to spray drying in a laboratory Spray. Dryer (Model Buchi-290) with a solution feed rate of 10 mL/min and an inlet temperature at 70° C. to yield an amorphous empagliflozin complex.

Yield: 14.5 g

### Example 6

Empagliflozin (10 g) was dissolved in methanol (200 mL) at 60±5° C. and Hydroxypropyl β-cyclodextrin (3.4 g) was added at 50±5° C. The reaction mass was maintained at 50±5° C. for 10 minutes to yield a clear solution. After cooling to 25±5′C, the resulting clear solution was filtered through hyflo to remove any undissolved particulate and subjected to spray drying in a laboratory Spray Dryer (Model Buchi-290) with a solution feed rate of 10 mL/min and an inlet temperature at 70° C. to yield an amorphous empagliflozin complex.

Yield: 7.3 g

### Example 7

Empagliflozin (10 g) was dissolved in methanol (200 mL) at 60±5° C. In another flask—β-cyclodextrin (1.1 g) was dissolved in water (90 mL) at 255° C. and the obtained solution was added to the empagliflozin solution at 25±5° C. The resulting clear solution was filtered through hyflo to remove any undissolved particulate and subjected to spray drying in a laboratory Spray Dryer (Model Buchi-290) with a solution feed rate of the 10 mL/min and an inlet temperature at 70° C. to yield amorphous empagliflozin complex.

Yield: 6.2 g

### Example 8

Empagliflozin (10 g) was dissolved in methanol (200 mL) at 60±5° C. In another flask—β-cyclodextrin (3.3 g) was dissolved in water (250 mL) at 25±5° C. and the obtained solution was added to the empagliflozin solution at 25±5° C. The resulting clear solution was filtered through hyflo to remove any undissolved particulate and subjected to spray drying in a laboratory Spray Dryer (Model Buchi-290) with solution feed rate of 10 mL/min and an inlet temperature at 70° C. to yield amorphous empagliflozin complex.

Yield: 7.5 g

### Example 9

Empagliflozin (10 g) was dissolved in methanol (200 mL) at 60±5° C. and Povidone K-30 (1.1 g) was added at 50±5° C. and the reaction mass was maintained at 50±5° C. for 10 minutes to yield a clear solution. After cooling to 25±5° C., the resulting clear solution was filtered through hyflo to remove any undissolved particulate and subjected to spray drying in a laboratory Spray Dryer (Model Buchi-290) with a solution feed rate of 10 mL/min and an inlet temperature at 70° C. to yield amorphous empagliflozin solid dispersion.

Yield: 5.5 g

1. An amorphous empagliflozin complex with a cyclodextrin.

2. The complex of claim 1, wherein the cyclodextrin is β-cyclodextrin or hydroxypropyl-β-cyclodextrin.

3. The complex of claim 1, wherein the cyclodextrin is β-cyclodextrin.

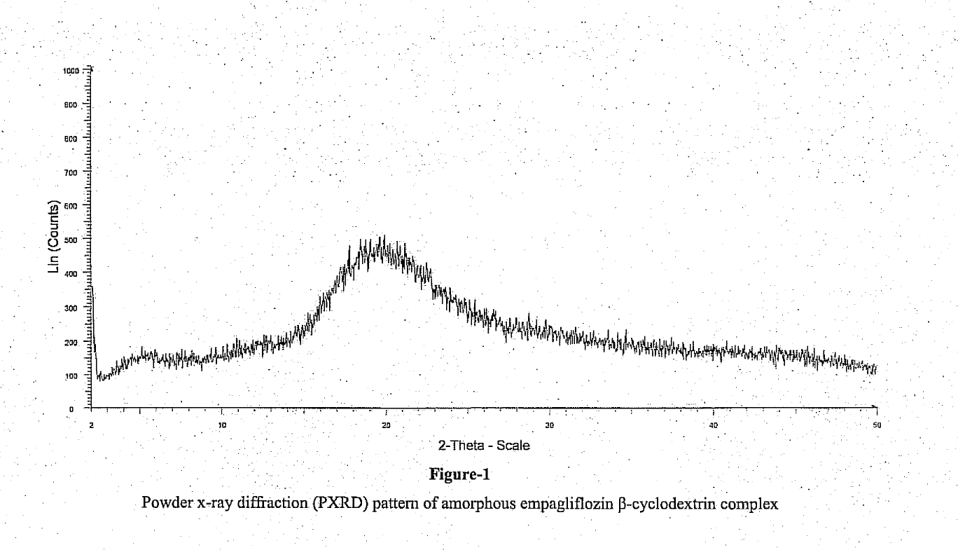

4. The complex of claim 3, having a powder X-ray diffraction (PXRD) pattern as shown in FIG. 1.

5. The complex of claim 1, wherein the cyclodextrin is hydroxypropyl-β-cyclodextrin.

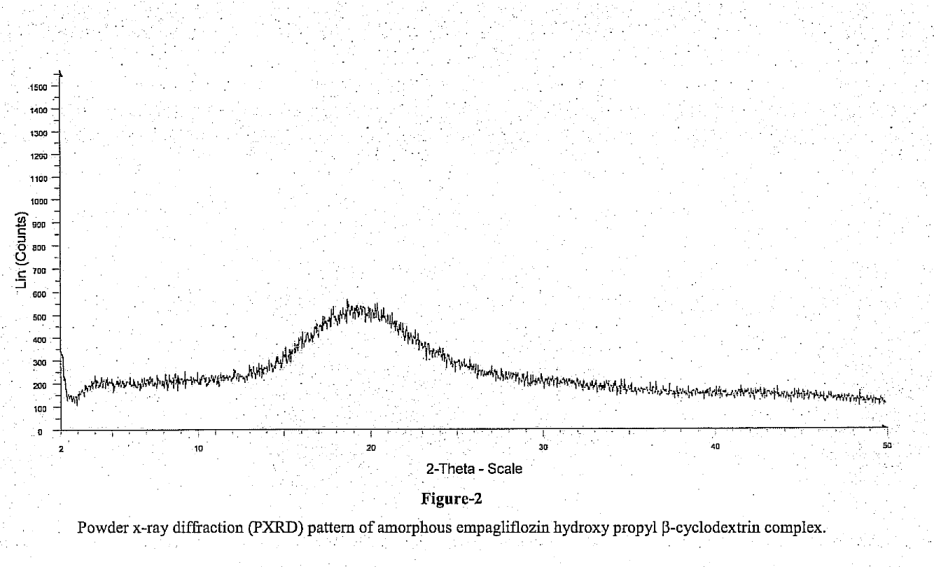

6. The complex of claim 5, having a powder X-ray diffraction (PXRD) pattern as shown in FIG. 2.

7. The complex of claim 1, wherein the amorphous empagliflozin complex comprises empagliflozin and the cyclodextrin in a weight ratio of about 20:1 to about 1:1.

8. The complex of claim 1, wherein the amorphous empagliflozin complex comprises empagliflozin and the cyclodextrin in a weight ratio of about 10:1 to about 2:1.

9. A process for preparing an amorphous empagliflozin complex comprising the steps of:
a) dissolving empagliflozin in a solvent to provide a first solution;
b) adding a cyclodextrin to the first solution; and
c) isolating an amorphous empagliflozin solid dispersion.

10. The process for preparing an amorphous empagliflozin complex of claim 9 further comprising the step of:
dissolving the cyclodextrin in water to provide a second solution prior to step b);
wherein step b) comprises combining the first solution and the second solution to provide a combined solution.

11. The process according to claim 9, wherein the cyclodextrin is selected from the group consisting of α-cyclodextrin, β-cyclodextrin, γ-cyclodextrin, and hydroxypropyl-β-cyclodextrin.

12. The process according to claim 9, wherein the cyclodextrin is selected from the group consisting of β-cyclodextrin and hydroxypropyl-β-cyclodextrin.

13. The process according to claim 9, wherein the cyclodextrin is β-cyclodextrin.

14. The process according to claim 9, wherein the cyclodextrin is hydroxypropyl-β-cyclodextrin.

15. The process according to claim 9, wherein the solvent is selected from the group consisting of methanol, ethanol, isopropanol, n-butanol, sec-butanol, 2-butanol, t-butanol, pentanol, and mixtures thereof.

16. The process according to claim 9, wherein the isolating is via by evaporation, distillation, spray drying, filtration, lyophilization, or an agitated thin film drier (ATFD).

17. The process of claim 9, wherein the empagliflozin and the cyclodextrin are present in a weight ratio of about 20:1 to about 1:1.

18. The process of claim 9, wherein the empagliflozin and the cyclodextrin are present in a weight ratio of about 10:1 to about 2:1.

19. A pharmaceutical composition comprising an amorphous cyclodextrin empagliflozin complex of claim 1 and a pharmaceutically acceptable excipient.

20. The composition of claim 19, comprising about 5 mg to about 50 mg of empagliflozin.

21. The composition of claim 19, further comprising a second antihyperglycemic agent.

22. The composition of claim 21, wherein the second antihyperglycemic agent is a dipeptidyl peptidase-4 (DPP-4) inhibitor.

23. The composition of claim 22, wherein the DPP-4 inhibitor is sitagliptin, vildagliptin, saxagliptin, linagliptin, anagliptin, teneligliptin, alogliptin, trelagliptin, gemigliptin, or dutogliptin.

24. The composition of claim 23, wherein the DPP-4 inhibitor is linagliptin.

25. The composition of claim 24, comprising 5 mg linagliptin.

26. The composition of claim 21, wherein the second antihyperglycemic agent is metformin hydrochloride.

27. The composition of claim 26, comprising about 500-1000 mg of metformin hydrochloride.

28. A method for improving glycemic control in a patient with type 2 diabetes mellitus, comprising administering to a patient in need thereof, a composition according to claim 1.
